(19) United States
(12) Patent Application Publication
(10) Pub. No.: US 2012/0065604 A1
(43) Pub. Date: Mar. 15, 2012

(54) DEGRADABLE HEMOSTATIC SPONGE AND EXTRUSION SYSTEM AND METHOD FOR MANUFACTURING THE SAME

(76) Inventors: Chao-Fu Chang, Chung-Li (CN); Kent Kuohua Chang, Cupertino, CA (US)

(21) Appl. No.: 12/880,424

(22) Filed: Sep. 13, 2010

(51) Int. Cl. A61L 15/64; B29C 44/00; A61F 13/36; B29C 44/04

(52) U.S. Cl. 604/368; 425/4 R; 264/28; 604/369

(57) ABSTRACT

A degradable hemostatic sponge that can be self-degraded and absorbed by a human body has poly lactic acid as its main material and mixed with a moisture-absorbent material, such as collagen, chitosan, starch and the like, at a specific ratio. Given grinding, mixing and melting steps, the materials using a supercritical fluid as a foaming agent can be used to manufacture the degradable hemostatic sponge having an open-cell microcellular form by a continuous extrusion foaming process. In addition, the present invention also includes a system and a method for manufacturing the degradable hemostatic sponge.

### BACKGROUND OF THE INVENTION

1. Field of the Invention

The present invention is related to a hemostatic sponge, and more particularly to a degradable hemostatic sponge and an extrusion system and method for manufacturing the same.

2. Description of the Related Art

For sake of small wound and surgical location deep into human body, the minimally invasive surgery usually has difficulty in stopping bleeding after the surgery. Given the nasal cavity operation as an example, post-operative hemostasis is performed by directly covering a wound with a hemostatic plate or a hemostatic sponge to press and stop bleeding of blood capillaries, veins and arteriosclerosis.

After 2 to 3 days a wound stops bleeding or recovers, such type of hemostasis requires to remove the hemostatic device inserted in the human body and oftentimes causes second injury of the wound and makes the recovery time and pain of the patient last longer.

With reference to FIG. 3, a conventional degradable hemostatic sponge may be made from polyurethane, polyether ester, ethyl carbamate or the like and is manufacture by a batch foaming process. With reference to FIG. 4, a bubble diameter of a conventional degradable hemostatic sponge is approximately 100 μm. With reference to FIG. 5, a degradation rate of a conventional degradable hemostatic sponge is about 7 days. With reference to FIG. 6, a conventional degradable hemostatic sponge degrades over time.

The conventional degradable hemostatic sponges have the following drawbacks:
1. As the conventional degradable hemostatic sponges are made from single polymer material, moisture absorption and degradation rate thereof are completely dominated by properties of materials in use. Therefore, moisture absorption and degradation rate fails to be further enhanced.
2. The bubble diameter of the conventional degradable hemostatic sponge is too large to have balanced pressure acted upon wound and blood capillary and further affecting the pressing effect and hemostasis. On the other hand, the oversized bubble diameter also results in lower capillary absorption of moisture, which causes insufficient water absorption for the conventional degradable hemostatic sponge to degrade. Moreover, the absorbed water that is unevenly distributed further slows down the degradation rate.

As far as the manufacturing method of the conventional degradable hemostatic sponge is concerned, there are the following shortcomings:
1. As the foaming agent for manufacturing the conventional degradable hemostatic sponge in a batch-foaming manner pertains to a regular chemical foaming agent, the manufactured conventional degradable hemostatic sponge requires two sterilization processes to comply with sanitary regulations of medical supplies.
2. Also because of the batch-foaming production, the foaming material requires a dipping process in the foaming agent. Such requirement results in a lengthy production time and the resulting foam porosity is not evenly distributed.

### SUMMARY OF THE INVENTION

A first objective of the present invention is to provide a degradable hemostatic sponge capable of absorbed by a human body.

To achieve the foregoing objective, the degradable hemostatic sponge has a foaming material. The foaming material has a foamable poly lactic acid (PLA) and an auxiliary moisture-absorbent material.

The foamable PLA is bio-degradable, is adapated to be absorbed by a human body and has an open-cell microcellular form with a bubble diameter less than 100 μm. The auxiliary moisture-absorbent material is selected from a group consisting of collagen, chitosan and starch, and is mixed with the foamable PLA.

The degradable hemostatic sponge of the present invention employs a foaming material having PLA affected by temperature thereof and moisture content therein and being degradable. After being added with an auxiliary moisture-absorbent material, such as collagen, chitosan, starch and the like, the foaming material can accelerate absorption of blood or water and a resulting degradation rate. The degradable hemostatic sponge of the present invention in possession of the PLA taking an open-cell microcellular form and a degradable characteristic can absorb blood, body fluid and moisture to develop a mildly and uniformly expansive pressure so as to demonstrate the hemostatic function. Moreover, the degradable hemostatic sponge can be degraded and absorbed by human body without having to remove it from patient's body, thereby avoiding second injury to patients after a surgery.

A second objective of the present invention is to provide an extrusion system for manufacturing the degradable hemostatic sponge.

To achieve the foregoing objective, the extrusion system has a power unit, an extrusion unit, a feed unit, a supercritical fill unit and an extrusion die.

The power unit has a mixing extrusion screw, a motor and a torque limiting device. The mixing extrusion screw has a pitch and geometric threads. The motor supplies power to rotate the mixing extrusion screw. The torque limiting device is connected with the motor and the mixing extrusion screw.

The extrusion unit has a mixing block defining a mixing chamber to receive the mixing extrusion screw.

The feed unit is connected and communicates with the extrusion unit and has a storage device and a feeder. The storage device has a foaming material agitation motor and a foaming material tank. The feeder is connected and communicates with the mixing block and has a feed screw and a feeder motor. The feeder motor supplies power to rotate the feed screw.

The supercritical fill unit has a foaming agent tank, a freezing and pressurizing device, a foaming agent pipe and a filling pipe.

The freezing and pressurizing device has a freezer and a high-pressure pump. The foaming agent pipe is connected and communicates with the foaming agent tank. The filling pipe is connected and communicates with the mixing block. The extrusion die is connected and communicates with the extrusion unit and has a pressure accumulation and discharge passageway.

A third objective of the present invention is to provide a method for manufacturing the degradable hemostatic sponge.

To achieve the foregoing objective, the method has steps of:

freezing and pressurizing a foaming agent to make the foaming agent reach a supercritical condition and become a supercritical foaming agent;

mixing and melting the supercritical foaming agent and a foaming material to form a liquid foaming material, wherein the supercritical foaming agent melted in the liquid foaming material forms bubbles in the liquid foaming material; and

accumulating pressure and discharging pressure of the liquid foaming material, wherein the bubbles grow for sake of an instant pressure drop when discharging pressure and form bubble chambers located inside the liquid foaming material and communicating with each other to achieve a foaming effect and acquire a degradable hemostatic sponge having a form of an open-cell microcellular foam.

The method manufacturing the degradable hemostatic sponge in accordance with the present invention, which utilizes the foaming material, such as the foamable PLA and the auxiliary moisture-absorbent material, in collaboration with the frozen and pressurized supercritical foaming agent to form the degradable hemostatic sponge having PLA taking an open-cell microcellular form by a continuous extrusion foaming process, indeed improves the conventional method for manufacturing degradable hemostatic sponge.

### BRIEF DESCRIPTION OF THE DRAWINGS

FIG. 1 is an image showing a detailed structure of a degradable hemostatic sponge in accordance with the present invention;

FIG. 2 is a schematic view of an extrusion system for manufacturing the degradable hemostatic sponge;

FIG. 3 is an image of a conventional degradable hemostatic sponge;

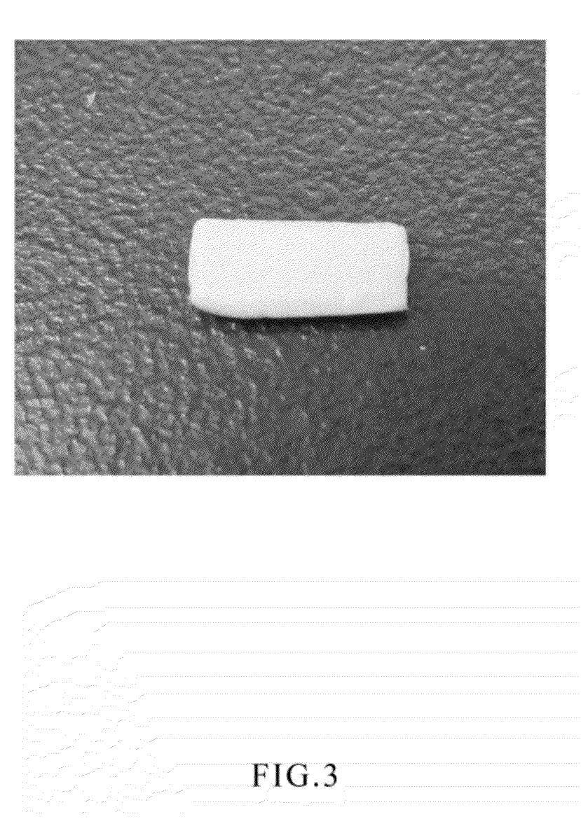

FIG. 4 is an image showing a detailed structure of the conventional degradable hemostatic sponge;

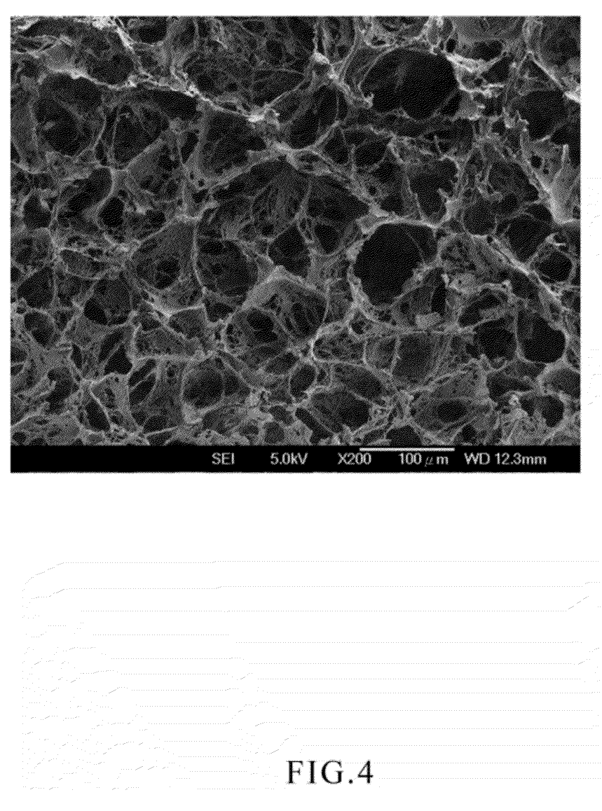

FIG. 5 is a chart showing degradation rate of the conventional degradable hemostatic sponge; and

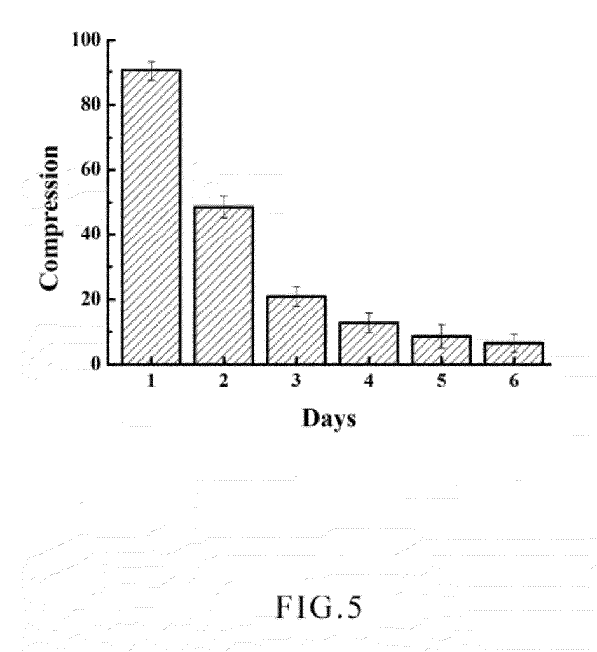

FIG. 6 is an image showing a degraded conventional degradable hemostatic sponge.

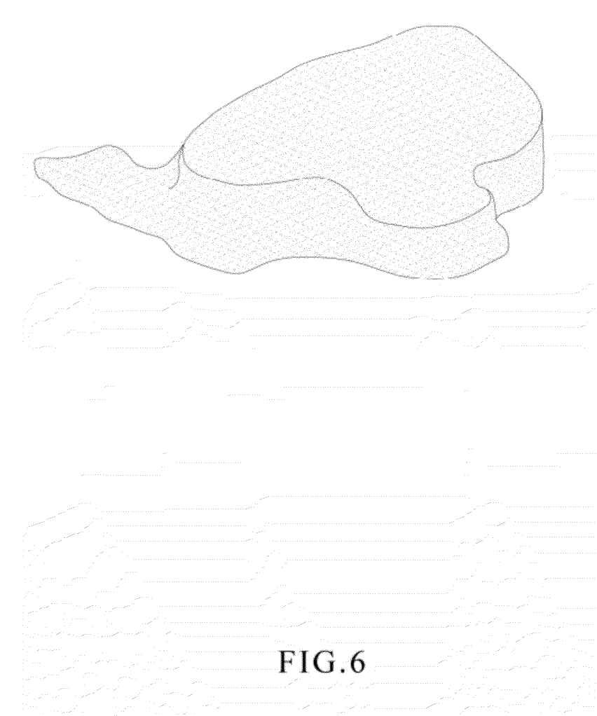

### DETAILED DESCRIPTION OF THE INVENTION

An embodiment of the degradable hemostatic sponge in accordance with the present invention exemplifies specific content thereof and demonstrates a special effect achieved by an open-cell microcellular form thereof.

With reference to Table 1, the degradable hemostatic sponge has a content composed of foamable poly lactic acid (PLA) and auxiliary moisture-absorbent material. The foamable PLA is bio-degradable and can be absorbed by human body. The auxiliary moisture-absorbent material is mixed with the foamable PLA at a specific ratio. The auxiliary moisture-absorbent material may be collagen, chitosan, starch or the like capable of increasing moisture absorption inside the degradable hemostatic sponge and accelerating the degradation rate.

TABLE 1

| | | | Percent of |
|---|---|---|---|
| | | Auxiliary | auxiliary |
| | | moisture- | moisture-absorbent |
| | Foamable | absorbent | material in overall |
| Item | PLA | material | materials |
| 1 | | Collagen | 25-50 |
| 2 | | Chitosan | 30-45 |
| 3 | | Starch | 15-45 |

The degradable hemostatic sponge of the present invention at least has the following advantages:
1. By mixing the bio-degradable foamable PLA with the auxiliary moisture-absorbent material, such as collagen, chitosan or starch, the degradable hemostatic sponge has a faster absorption of blood or water in favor of higher moisture content in the degradable hemostatic sponge and faster degradation rate thereof.
2. As the foamable PLA takes an open-cell microcellular form, the pressure transferred by the attached degradable hemostatic sponge can be uniformly distributed across wounds or blood capillaries and beneficial to the pressing effect and hemostasis on wound in the early stage.

A method for manufacturing degradable hemostatic sponge in accordance with the present invention exemplifies a substantial method of producing the degradable hemostatic sponge of the present invention and a special effect gained from the method.

The method for manufacturing degradable hemostatic sponge has the following steps:

freezing and pressurizing a foaming agent to make the foaming agent reach a supercritical condition and become a supercritical foaming agent; mixing and melting the supercritical foaming agent and a foaming material to form a liquid foaming material. The supercritical foaming agent melted in the liquid foaming material forms bubbles in the liquid foaming material; and accumulating pressure and discharging pressure of the liquid foaming material; when discharging pressure, the bubbles grow for sake of an instant pressure drop and form bubble chambers located inside the foaming material and communicating with each other to achieve a foaming effect and acquire a degradable hemostatic sponge having a form of an open-cell microcellular foam.

In addition, the operation temperature preferably ranges from 100° C. to 170° C. when performing the mixing and melting step.

Nitrogen or carbon dioxide may be used as the foaming agent. Those gases serving as the foaming agent do not generate any chemical reaction with the foaming material, and thus will not contaminate the foaming material. Accordingly, the manufactured degradable hemostatic sponge is immune to second sterilization.

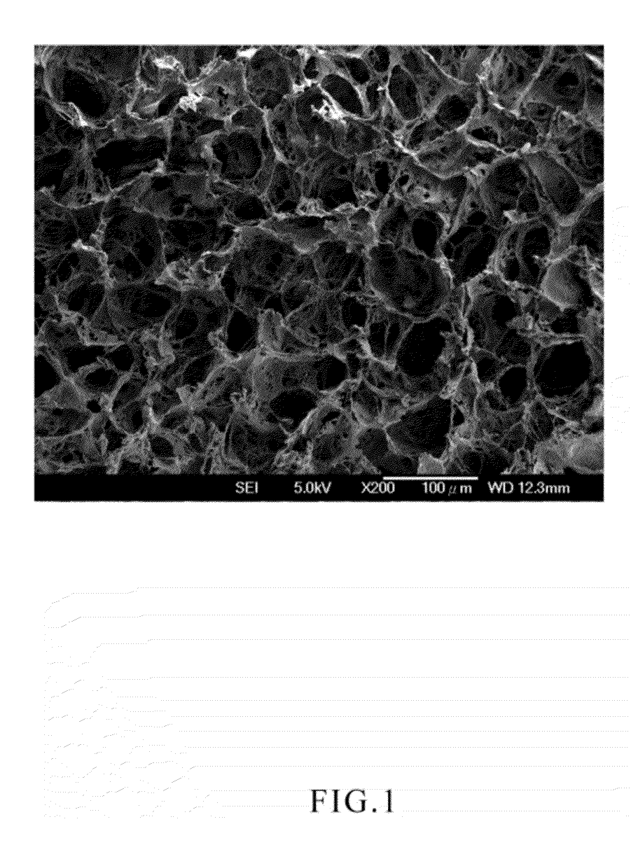

The method for manufacturing degradable hemostatic sponge employs a continuous microcellular supercritical fluid extrusion foaming technique having at least the following advantages:
1. Producing the degradable hemostatic sponge with the continuous extrusion foaming method can accelerate production speed and save production cost, thereby effectively lowering the selling price of the degradable hemostatic sponge and increasing the competitive edge in the commercial market.
2. The foaming agent is frozen to form a supercritical foaming agent to serve as a physical foaming agent that generates no chemical change to the foaming material. Besides, the supercritical foaming agent is capable of detoxify the foaming material.
3. The supercritical foaming agent collaborated with a continuous foam extrusion system can control a bubble diameter in the manufactured foam having an open-cell microcellular form, which is lower than 100 μm. With reference to FIG. 1, under normal circumstance the bubble diameter can be controlled down to 90 μm and below, thereby increasing the moisture and accelerating the degradation rate.

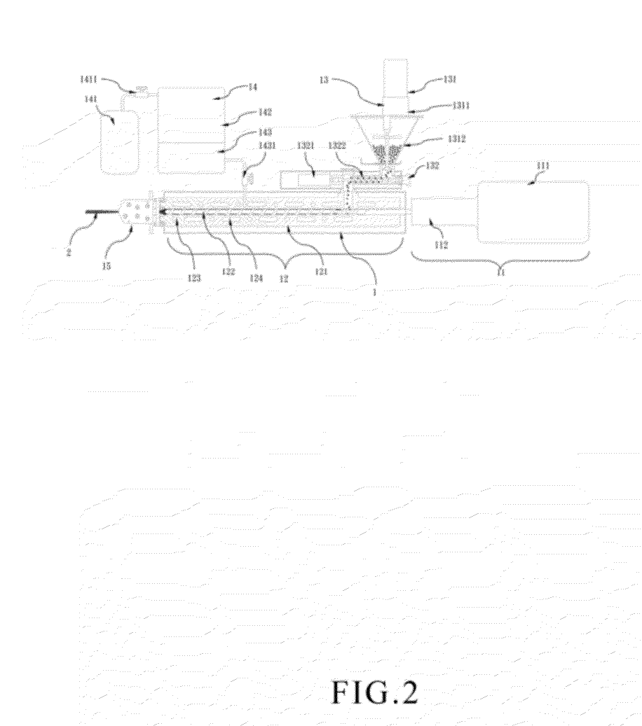

With reference to FIG. 2, an extrusion system for manufacturing degradable hemostatic sponge in accordance with the present invention exemplifies the use of the system to realize the aforementioned method for manufacturing the degradable hemostatic sponge and to manufacture the degradable hemostatic sponge of the present invention.

The extrusion system for manufacturing degradable hemostatic sponge has a power unit 11, an extrusion unit 12, a feed unit 13, a supercritical fill unit 14 and an extrusion die 15.

The power unit 11 serves to supply power to operate the extrusion system and has a mixing extrusion screw 122, a motor 111 and a torque limiting device 112. The mixing extrusion screw 122 has a special pitch and geometric threads. The motor 111 supplies power to rotate the mixing extrusion screw 122. The torque limiting device 112 is connected with the motor 111 and the mixing extrusion screw 122, and serves for protection purpose to prevent from generating excessively large torque and prevent the motor 111 or the mixing extrusion screw 122 from being overloaded.

The extrusion unit 12 serves to feed, grind, melt, mix, press and extrude a foaming material. The extrusion unit 12 has a mixing block 121, a heating device 123 and a cooling device 124. The mixing block 121 receives the mixing extrusion screw 122 and has a mixing chamber defined therein for mixing the foaming material and being kept at a constant temperature so that the foaming material conveyed to the mixing block 121 can be mixed in a specified temperature range or at a specified temperature. The heating device 123 is mounted outside the mixing block 121 to maintain the temperatures of the mixing block 121 and the mixing chamber so as to facilitate the mixing of foaming material at a constant temperature. The cooling device 124 is equipped to adjust the temperatures of the mixing block 121 and the mixing chamber in collaboration with the heating device 123. A gaseous or liquid refrigerant circulates inside the cooling device 124 to achieve a cooling effect so as to adjust or balance the temperatures of the mixing block 121 and the mixing chamber.

The feed unit 13 serves to feed the foaming material and is connected and communicates with the extrusion unit 12. The feed unit 13 has a storage device 131 and a feeder 132. The storage device 131 has a foaming material agitation motor 1311 and a foaming material tank 1312. The foaming material agitation motor 1311 serves to supply power to agitate the foaming material. The foaming material tank 1312 serves to store the foaming material. The feeder 132 is connected and communicates with the mixing block 121 and has a feed screw 1322 and a feeder motor 1321. The feed screw 1322 serves to propel and convey the foaming material. The feeder motor 1321 supplies power to rotate the feed screw 1322.

The supercritical fill unit 14 serves to supply the supercritical foaming agent and has a foaming agent tank 141, a freezing and pressurizing device, a foaming agent pipe 1411 and a filling pipe 1431. The freezing and pressurizing device has a freezer 142 and a high-pressure pump 143. The foaming agent pipe 1411 is connected and communicates with the foaming agent tank 141. The filling pipe 1431 is connected and communicates with the mixing block 121. The freezing and pressurizing device serves to freeze and pressurize the foaming agent to form a supercritical foaming agent after the foaming agent reaches a supercritical condition. The supercritical foaming agent is filled in the mixing block 121 through the filling pipe 1431 to mix and melt with the foaming material, and the mixed and melted supercritical foaming agent and the foaming material forms a liquid foaming material as a whole. To facilitate to control a flow rate of the foaming agent, the foaming agent pipe 1411 has a control valve and the filling pipe 1431 has a valve.

The extrusion die 15 is connected and communicates with the extrusion unit 12 and has a pressure accumulation and discharge passageway. The extrusion die 15 receives the liquid foaming material from the extrusion unit 12. The pressure accumulation and discharge passageway inside the extrusion die 15 is a flow channel designed specifically to accumulate and discharge pressure of the melted liquid foaming material so as to achieve a foaming effect. Given the pressure accumulation and discharge processing, when the supercritical foaming agent dissolved and forming bubbles in the liquid foaming material undergoes the pressure discharge region of the extrusion die 15, the bubbles grow and form the bubble chambers inside the liquid foaming material due to sudden pressure drop arising from enlarged space in the flow channel. The bubble chambers can communicate with each other, thereby acquiring a degradable hemostatic sponge demonstrating an open-cell microcellular form.

The mixing ratio of the foamable PLA and the auxiliary moisture-absorbent material mentioned in the foregoing description and a corresponding critical manufacturing condition used to produce the degradable hemostatic sponge are provided as follows:
1. Mixing ratio of PLA and Collagen: 75:25 to 50:50;

Extrusion temperature of extrusion die: 105° C. to 125° C.;

Pressure of extrusion die: 50 bar to 75 bar;

Foaming agent: Nitrogen;

Filling pressure: 300 bar to 400 bar.
2. Mixing ratio of PLA and Chitosan: 70:30 to 55:45;

Extrusion temperature of extrusion die: 115° C. to 125° C.;

Pressure of extrusion die: 50 bar to 75 bar;

Foaming agent: Nitrogen;

Filling pressure: 300 bar to 400 bar.
3. Mixing ratio of PLA and starch: 85:15 to 55:45;

Extrusion temperature of extrusion die: 115° C. to 125° C.;

Pressure of extrusion die: 50 bar to 75 bar;

Foaming agent: Nitrogen;

Filling pressure: 300 bar to 400 bar.

With reference to Table 2, by comparing conventional degradable hemostatic sponge and the degradable hemostatic sponge of the present invention, it is unambiguous to understand the special effect provided by the technical means of the present invention.

TABLE 2

| | Conventional | Degradable hemostatic |
|---|---|---|
| | degradable | sponge of the |
| Content | hemostatic sponge | present invention |
| Foaming | Single material: | Combined materials: |
| material | Polyurethane | PLA + collagen |
| | | PLA + chitosan |
| | | PLA + starch |
| Foaming | Batch-foaming | Continuous extrusion |
| method | | foaming |
| Foaming | Chemical/Physical | Physical (supercritical |
| agent | | foaming agent) |
| Bubble | More than 100 μm | Less than 100 μm |
| diameter | | |
| Structural | Weaker | Stronger |
| strength | | |
| Capillary | Slower | Faster |
| velocity | | |
| Pressurization | Weaker | Stronger |
| Moisture | Lower | Higher |
| content of sponge | | |
| Degradation | Slower | Faster |
| rate | | |

In view of the foregoing comparison, the present invention indeed improves the drawbacks of the conventional technique. Moreover, the technical means provided by the present invention is suitable for mass production of degradable hemostatic sponge and effectively increases the production capacity of the degradable hemostatic sponge having the self-degradability and the open-cell micro cellular form.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A degradable hemostatic sponge, comprising a foaming material having: a foamable poly lactic acid (PLA) being bio-degradable, adapted to be absorbed by a human body and having an open-cell microcellular form with a bubble diameter less than 100 μm; and an auxiliary moisture-absorbent material selected from a group consisting of collagen, chitosan and starch, and mixed with the foamable PLA.

2. The degradable hemostatic sponge as claimed in claim 1, wherein the auxiliary moisture-absorbent material is collagen; and a mixing ratio of the foamable PLA and the auxiliary moisture-absorbent material ranges from 75:25 to 50:50.

3. The degradable hemostatic sponge as claimed in claim 1, wherein the auxiliary moisture-absorbent material is chitosan; and a mixing ratio of the foamable PLA and the auxiliary moisture-absorbent material ranges from 70:30 to 55:45.

4. The degradable hemostatic sponge as claimed in claim 1, wherein the auxiliary moisture-absorbent material is starch; and a mixing ratio of the foamable PLA and the auxiliary moisture-absorbent material ranges from 85:15 to 55:45.

5. An extrusion system for manufacturing degradable hemostatic sponge, comprising:
a power unit having:
a mixing extrusion screw having a pitch and geometric threads;
a motor supplying power to rotate the mixing extrusion screw; and
a torque limiting device connected with the motor and the mixing extrusion screw;
an extrusion unit having a mixing block defining a mixing chamber to receive the mixing extrusion screw;
a feed unit connected and communicating with the extrusion unit and having:
a storage device having a foaming material agitation motor and a foaming material tank; and
a feeder connected and communicating with the mixing block and having:
a feed screw; and
a feeder motor supplying power to rotate the feed screw;
a supercritical fill unit having:
a foaming agent tank;
a freezing and pressurizing device having a freezer and a high-pressure pump;
a foaming agent pipe connected and communicating with the foaming agent tank; and
a filling pipe connected and communicating with the mixing block; and
an extrusion die connected and communicating with the extrusion unit and having a pressure accumulation and discharge passageway.

6. The extrusion system as claimed in claim 5, wherein the foaming agent pipe has a control valve and the filling pipe has a valve.

7. The extrusion system as claimed in claim 5, wherein the extrusion system further comprises:
a heating device mounted outside the mixing block; and
a cooling device balancing temperatures of the mixing block and the mixing chamber.

8. The extrusion system as claimed in claim 7, wherein a refrigerant circulates inside the cooling device and is gaseous or liquid.

9. A method for manufacturing degradable hemostatic sponge, comprising steps of:
freezing and pressurizing a foaming agent to make the foaming agent reach a supercritical condition and become a supercritical foaming agent;
mixing and melting the supercritical foaming agent and a foaming material to form a liquid foaming material, wherein the supercritical foaming agent melted in the liquid foaming material forms bubbles in the liquid foaming material; and
accumulating pressure and discharging pressure of the liquid foaming material, wherein the bubbles grow for sake of an instant pressure drop when discharging pressure, and form bubble chambers located inside the liquid foaming material and communicating with each other to achieve a foaming effect and acquire a degradable hemostatic sponge having a form of an open-cell microcellular foam.

10. The method as claimed in claim 9, wherein the mixing and melting step is performed under a temperature ranging from 100° C. to 170° C.

11. The method as claimed in claim 9, wherein the foaming agent is nitrogen or carbon dioxide.

12. The method as claimed in claim 9, further comprising a step of providing an extrusion system for manufacturing degradable hemostatic sponge before freezing and pressurizing the foaming agent, wherein the extrusion system has:
a power unit having:
a mixing extrusion screw having a pitch and geometric threads;
a motor supplying power to rotate the mixing extrusion screw; and
a torque limiting device connected with the motor and the mixing extrusion screw;
an extrusion unit having:
a mixing block defining a mixing chamber to receive the mixing extrusion screw;
a heating device mounted outside the mixing block; and
a cooling device for balancing temperatures of the mixing block and the mixing chamber;
a feed unit connected and communicating with the extrusion unit and having:
a storage device having a foaming material agitation motor and a foaming material tank; and
a feeder connected and communicating with the mixing block and having:
a feed screw; and
a feeder motor supplying power to rotate the feed screw;
a supercritical fill unit having:
a foaming agent tank;
a freezing and pressurizing device having a freezer and a high-pressure pump;
a foaming agent pipe connected and communicating with the foaming agent tank; and
a filling pipe connected and communicating with the mixing block; and
an extrusion die connected and communicating with the extrusion unit and having a pressure accumulation and discharge passageway;
whereby
the freezer and the high-pressure pump of the freezing and pressurizing device freezes and pressurizes a foaming agent received in the foaming agent tank and conveyed through the foaming agent pipe to the freezing and pressurizing device to form a supercritical foaming agent when the foaming agent reaches a supercritical condition;
the supercritical foaming agent is filled in the mixing block and the foaming material tank received in the storage device of the feed unit through the filling pipe, and is outputted to the mixing block and mixed and melted with a foaming material by the feed screw of the feed unit to form a liquid foaming material; and
a pressure of the liquid foaming material is accumulated and discharged through the extrusion die so that bubbles that are formed by the supercritical foaming agent dissolved in the in the liquid foaming material grow when the pressure is discharged and instantly reduced, and form bubble chambers communicating with each other in the liquid foaming material to acquire a degradable hemostatic sponge having a form of an open-cell microcellular foam.

13. The method as claimed in claim 9, wherein the foaming material comprises:
a foamable PLA being bio-degradable, and adapted to be absorbed by a human body; and
an auxiliary moisture-absorbent material selected from a group consisting of collagen, chitosan and starch, and mixed with the foamable PLA.

14. The method as claimed in claim 10, wherein the foaming material comprises:
a foamable PLA being bio-degradable, and adapted to be absorbed by a human body; and
an auxiliary moisture-absorbent material selected from a group consisting of collagen, chitosan and starch, and mixed with the foamable PLA.

15. The method as claimed in claim 11, wherein the foaming material comprises:
a foamable PLA being bio-degradable, and adapted to be absorbed by a human body; and
an auxiliary moisture-absorbent material selected from a group consisting of collagen, chitosan and starch, and mixed with the foamable PLA.

16. The method as claimed in claim 12, wherein the foaming material comprises:
a foamable PLA being bio-degradable, and adapted to be absorbed by a human body; and
an auxiliary moisture-absorbent material selected from a group consisting of collagen, chitosan and starch, and mixed with the foamable PLA.

17. The method as claimed in claim 16, wherein
the foaming agent is nitrogen;
the supercritical fill unit fills the supercritical foaming agent with a filling pressure ranging from 300 bar to 400 bar;
the auxiliary moisture-absorbent material is collagen;
a mixing ratio of the PLA and the moisture-absorbent material ranges from 75:25 to 50:50; and
the extrusion die has an extrusion temperature ranging from 105° C. to 125° C. and a pressure of the extrusion die ranging form 65 bar to 75 bar.

18. The method as claimed in claim 16, wherein
the foaming agent is nitrogen;
the supercritical fill unit fills the supercritical foaming agent with a filling pressure ranging from 300 bar to 400 bar;
the auxiliary moisture-absorbent material is chitosan;
a mixing ratio of the PLA and the moisture-absorbent material ranges from 70:30 to 55:45; and
the extrusion die has an extrusion temperature ranging from 115° C. to 125° C. and a pressure of the extrusion die ranging form 50 bar to 75 bar.

19. The method as claimed in claim 16, wherein
the foaming agent is nitrogen;
the supercritical fill unit fills the supercritical foaming agent with a filling pressure ranging from 300 bar to 400 bar;
the auxiliary moisture-absorbent material is starch;
a mixing ratio of the PLA and the moisture-absorbent material ranges from 85:15 to 55:45; and
the extrusion die has an extrusion temperature ranging from 115° C. to 125° C. and a pressure of the extrusion die ranging form 50 bar to 75 bar.
